(19) United States
(12) Patent Application Publication
(10) Pub. No.: US 2003/0195391 A1
(43) Pub. Date: Oct. 16, 2003

(54) Lighting and unlocking instrument

(76) Inventor: Chel Ho Shin, Seoul (KR)

Correspondence Address: CHRISTIE, PARKER & HALE, LLP, 350 WEST COLORADO BOULEVARD, SUITE 500, PASADENA, CA, 91105, US

(21) Appl. No.: 10/124,620

(22) Filed: Apr. 16, 2002

(51) Int. Cl. A61B 1/267

(52) U.S. Cl. 600/200

(57) ABSTRACT

A lighting and unlocking instrument for achieving a quick and easy unlocking operation is disclosed. The unlocking instrument has a handle grip containing dry cells, a speculum body coupled to an upper end of the handle grip and including a magnifying lens, a coupler coupled to a front part of the speculum body, a rotating body fitted on the coupler and having a slot for holding a probing pick, and a fastening bolt screwed into the rotating body to fasten the rotating body to the coupler.

### BACKGROUND OF THE INVENTION

1. Field of the Invention

The present invention relates to a lighting and unlocking instrument, and more particularly to a lighting and unlocking instrument which is provided with a known magnifier for lighting a dark inside of a keyhole and for allowing the inside of the keyhole to be magnified and to be seen by a user, and which is also provided at the front of the magnifier with a probing pick capable of examining the inside of a keyhole such that the probing pick can be controlled in its mounting angle, thereby achieving quick and easy unlocking operation and manufacturing process of a key.

2. Description of the Prior Art

As is commonly known, a magnifier, which is designed to shine a small hole to allow the lighted area of the hole to be more clearly seen by a user, is broadly used in various fields.

In particular, a medical otoscope is used to examine an external canal and a tympanic membrane of an ear. As shown in FIG. 1, such a medical otoscope is comprised of a handle grip 2 containing dry cells and a speculum body 3 coupled to the top of the handle grip 2.

The speculum body 3 is provided internally with a bulb 4 and at a rear end thereof with a magnifying lens 5. The handle grip 2 includes an On-Off switch 6 at its middle portion, and the speculum body 3 is formed at its front portion with a coupling groove 3a to enable a speculum 8 to be coupled thereinto.

When a key is broken in a keyhole of a lock body or is lost after locking of a lock body due to user's carelessness, it is generally required to unlock the lock body while examining an arrangement of locking pins received into pinholes formed at the inner surface of the keyhole.

To this end, after a worker puts a magnifying lens over his eye, the worker must try to unlock the lock by a probing pick using his one hand, while lighting the inside of the keyhole by a flashlight using his other hand. However, since such an unlocking operation is very troublesome and difficult, time required to complete the unlocking operation is prolonged.

Accordingly, to solve the above problems, it is strongly required to provide a lighting and unlocking instrument which is integrally provided therein with various tools necessary to precisely examine a keyhole of a lock and to unlock the lock, thereby easily and conveniently fulfilling an unlocking operation, and resulting in maximization of operating efficiency.

The inventor recognized the fact that a conventional magnifier is employed in only a specific field, for example, a medical field, but not efficiently employed in an unlocking operation.

### SUMMARY OF THE INVENTION

Accordingly, the present invention has been made keeping in mind the above problems occurring in the prior art, and an object of the present invention is to provide a lighting and unlocking instrument which is integrally provided therein with various tools necessary for an unlocking operation, thereby easily and conveniently fulfilling an unlocking operation, and resulting in maximization of operating efficiency.

The lighting and unlocking instrument according to the present invention can unlock a lock after examination of an inside of a keyhole, and also enables a key mating the lock to be extemporarily manufactured according to the examination results.

In order to accomplish the above object, the present invention provides a lighting and unlocking instrument comprising: a handle grip containing dry cells therein; a speculum body coupled to an upper end of the handle grip and including a magnifying lens at its rear end; a coupler detachably coupled to a front part of the speculum body; a rotating body fitted on an outer surface of a front end of the coupler and having a holding slot into which a probing pick is securely inserted; and a fastening bolt screwed into the rotating body to fasten the rotating body to the coupler.

Accordingly, the lighting and unlocking instrument according to the present invention can unlock a lock after examination of protruding degrees and arrangement of locking pins arranged in a keyhole, and also enables a key mating the lock to be extemporarily manufactured.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a cross-sectional view of a conventional otoscope;

FIG. 2 is an exploded perspective view of a lighting and unlocking instrument according to the present invention;

FIG. 3 is a perspective view of a coupler assembled with a rotating body and a probing pick according to the present invention;

FIG. 4 is a cross-sectional view of the unlocking instrument according to the present invention;

FIG. 5 is view showing an unlocking operation using the unlocking instrument according to the present invention; and

FIGS. 6a to 6d are perspective views showing other probing picks of the present invention.

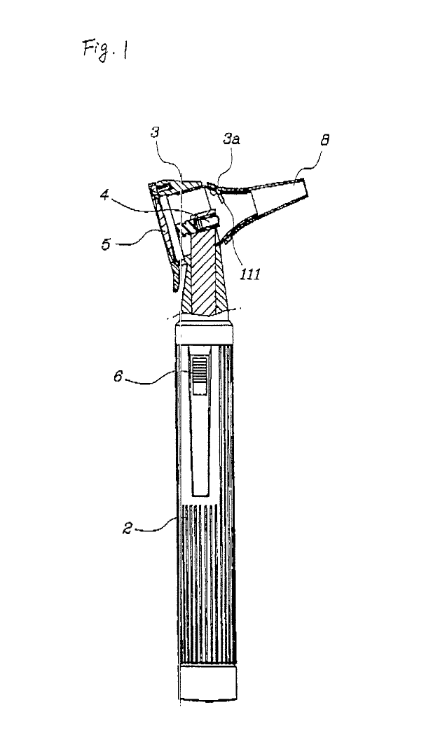

### DETAILED DESCRIPTION OF THE INVENTION

This invention will be described in further detail by way of example with reference to the accompanying drawings.

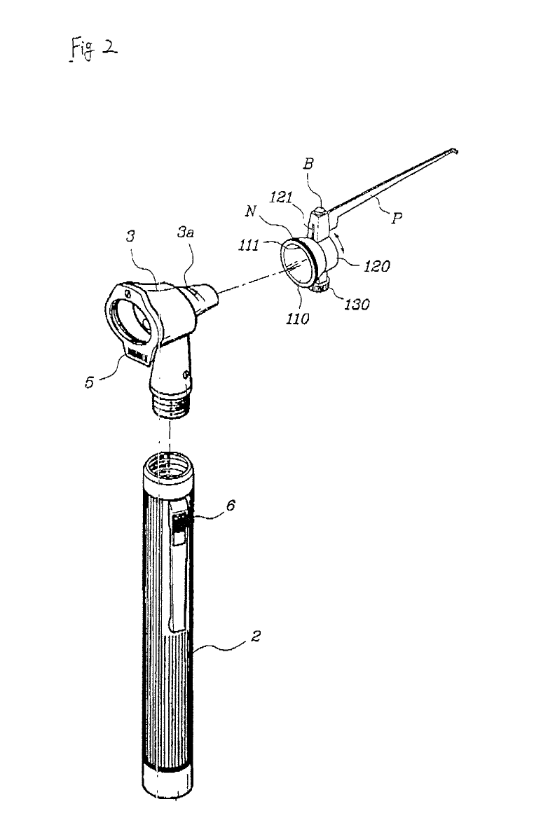

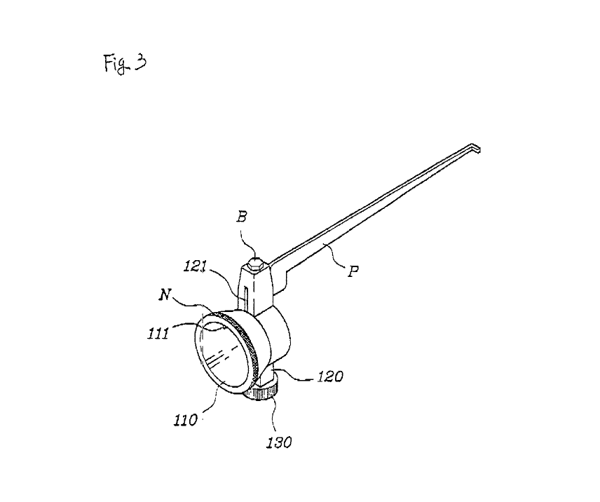

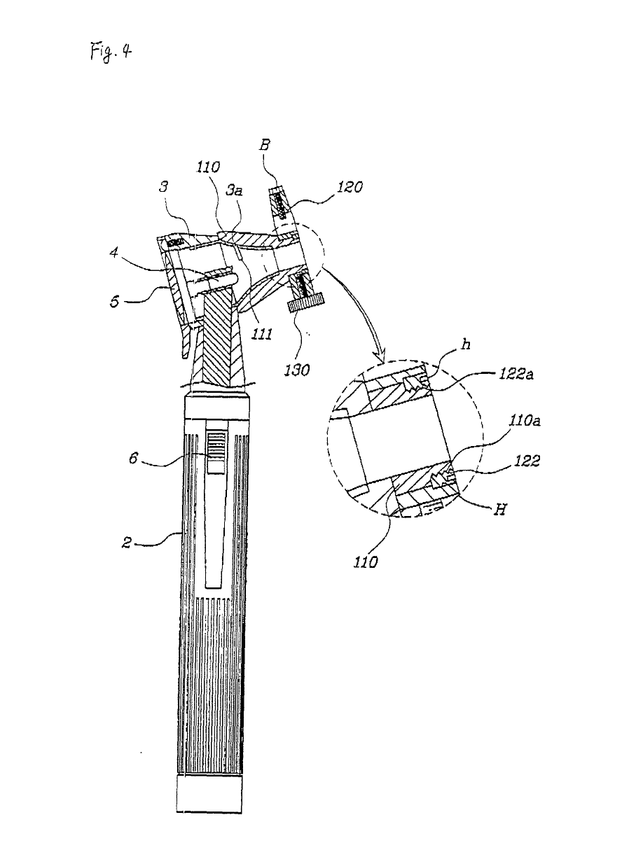

FIGS. 2 to 5 show a lighting and unlocking instrument according to the present invention. As shown in the drawings, the unlocking instrument of the present invention comprises a handle grip 2 containing dry cells (not shown) therein, a speculum body 3 coupled to an upper end of the handle grip 2 and including a magnifying lens 5 at its rear end, a coupler 110 detachably coupled to a front part of the speculum body 3, a rotating body 120 fitted on an outer surface of a front end of the coupler 110 and having a holding slot 121 into which a probing pick “P” is securely inserted, and a fastening bolt 130 screwed into the rotating body 120 to fasten the rotating body to the coupler.

More specifically, the handle grip 2 can contain dry cells therein, and is provided at its outer surface with the On-Off switch 6.

The speculum body 3 is coupled to the handle grip 2 such that the speculum body 3 is positioned with respect to the handle grip 2 with a certain angle therebetween. That is, the speculum body 3 is positioned to be approximately perpendicular to the handle grip 2.

The speculum body 3 is centrally provided with a bulb 4, and provided at its rear end with the magnifying lens 5. Furthermore, the speculum body 3 is formed at its front taper surface with a fitting groove 3a to allow the coupler 110 to be coupled thereinto. To this end, the coupler 110 is provided at its inner surface with a fitting protrusion 111. Accordingly, when the coupler 110 is fitted on the front taper surface of the speculum body 3 and then rotated with respect to the speculum body 3, the fitting protrusion 111 of the coupler 110 is engaged with the fitting groove 3a of the speculum body 3, thereby achieving firm coupling between the coupler 110 and the speculum body 3.

The coupler 110 is shaped into a hollow truncated circular cone, which becomes narrower frontwardly. Moreover, the coupler 110 is provided at outer surface of its rear end with an annular knurled section “N”, to enable a user to easily rotate the coupler 110.

The rotating body 120 is rotatably joined to a front part of the coupler 110, and is formed with a holding slot 121 in which the probing pick “P” is held. A holding bolt “B” is screwed into the coupler 120 and then protruded into the holding slot 121.

The holding slot 121 is formed at a proper position such that, when the probing pick “P” is held in the holding slot 121, the tip of the probing pick “P” can be seen through the magnifying lens 5. That is, only when the tip of the probing pick “P” can be seen through the magnifying lens 5, a user can easily and precisely carry out an unlocking operation.

A retaining ring 122 is interposed between the rotating body 120 and the coupler 110, which serves as a stopper for preventing the coupler 110 coupled to the rotating body 120 from being separated therefrom.

The coupler 110 is formed with a threaded portion 110a on a outer surface of its front end, and thus the retaining ring 122 is formed with a threaded portion 122a mating the threaded portion 110a of the coupler 110.

In other words, by fitting the rotating body 120 on the front end of the coupler 110, between the coupler 110 and the rotating body 120 is formed a ring groove “H”. The threaded portion 110a of the coupler 110 is exposed at the inner surface of the ring groove “H”, so that the threaded portion 122a of the retaining ring 122 can be engaged with the threaded portion 110a of the coupler 110 by rotating the retaining ring 122. The retaining ring 122 is formed at its outer end surface with tool grooves “h” to enable the retaining ring 122 to be easily rotated.

The rotating body 120 is provided at its lower end with the fastening bolt 130 to lock the rotating body 120 to the coupler 110. Therefore, after the rotating body 120 is rotated by a certain angle, the fastening bolt 130 is tightened to lock the rotating body 120 in place.

Assembling and unlocking operations of the unlocking instrument according to the present invention will now be described.

In an operation of coupling the coupler 110 and the rotating body 120 to the speculum body 3, as the coupler 110 is fitted on the speculum body 3 and then rotated in a certain direction, the fitting protrusion 111 of the coupler 110 is engaged with the fitting groove 3a of the speculum body 3, thereby achieving firm coupling of the speculum body 3 and the coupler 110.

Subsequently, the rotating body 120 is fitted on the coupler 110 to have the ring groove “H” therebetween, and the threaded portion 122a of the retaining ring 122 is engaged with the threaded portion 110a of the coupler 110, in order to prevent the rotating body 120 from being separated from the coupler 110. At this point, the retaining ring 122 can be easily rotated by means of the tool grooves “h”.

Thereafter, the probing pick “P” is inserted into the holding slot 121 of the rotating body 120, and the fastening bolt “B” is tightened to firmly hold the probing pick “P”. By so doing, the coupling operation is completed.

In an unlocking operation, a proper probing pick “P” is first inserted into the holding slot 121 of the rotating body 120 and then held therein by the holding bolt “B”. Subsequently, the rotating body 120 is rotated to a desired circumferential position by tightening or releasing the fastening bolt 130.

After the On-Off switch 6 is switched to ON to turn on the bulb 2, a user controls orientation of the speculum body 3 to direct a beam of light emitting from the speculum body 3 into a keyhole while gripping the handle grip, and then carries out an unlocking manipulation.

In addition to the unlocking operation, it is possible to immediately manufacture a key according to information obtained by the examination of arrangement and protruding degree of locking pins in the keyhole.

If it is desired to again adjust a mounting angle of the probing pick “P” because a space required for an unlocking operation is restricted or work posture is unstable, the fastening bolt 130 is released and the rotating body 120 is rotated to adjust the mounting angle, followed by tightening of the fastening bolt 130.

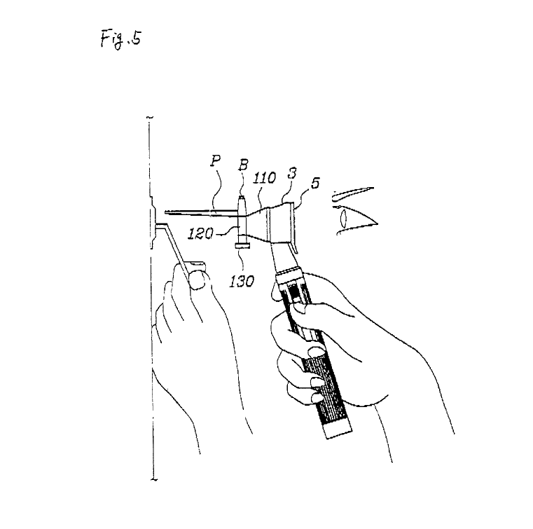

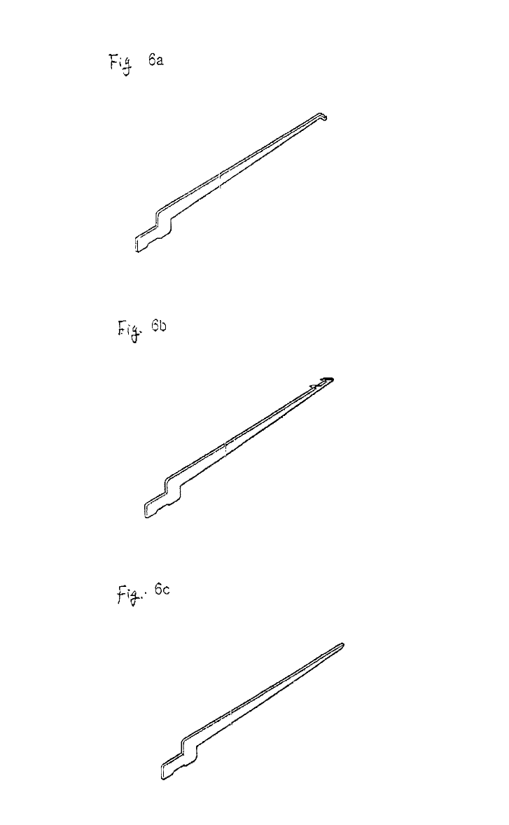

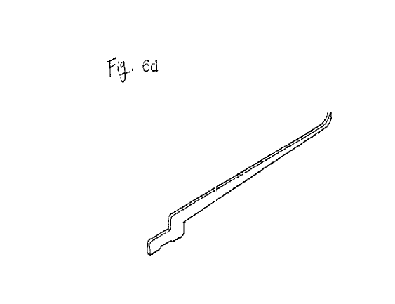

As shown in FIGS. 6a to 6d, the unlocking instrument according to the present invention may employ other probing picks if required. More specifically, FIG. 6a shows a probing pick applicable to a lock for an automobile, FIG. 6b shows a pick for extracting a broken key piece from a keyhole, FIG. 6c shows a probing pick for identifying locking pins in a keyhole, and FIG. 6d shows a probing pick used as an auxiliary tool.

As described above, the present invention provides a lighting and unlocking instrument, which is provided with a usual magnifier for lighting a dark inside of a keyhole and for allowing the inside of the keyhole to be magnified and to be seen by a user, and which is also provided with a probing pick at the front of the magnifier such that the probing pick can be controlled in its mounting angle, thereby achieving quick and easy unlocking operation and manufacture of a key.

The lighting and unlocking instrument according to the present invention can be applied to all kinds of locks such as particular locks for automobiles and locks of doors of various buildings.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A lighting and unlocking instrument comprising:
a handle grip containing dry cells therein;
a speculum body coupled to an upper end of the handle grip and including a magnifying lens at its rear end;
a coupler detachably coupled to a front part of the speculum body;
a rotating body fitted on an outer surface of a front end of the coupler and having a holding slot into which a probing pick is securely inserted; and
a fastening bolt screwed into the rotating body to fasten the rotating body to the coupler.
